(19) United States
(12) Patent Application Publication
(10) Pub. No.: US 2011/0092869 A1
(43) Pub. Date: Apr. 21, 2011

(54) ARTICLES HAVING ZONED BREATHABILITY

(75) Inventors: John Chu Chen, Hockessin, DE (US); Donna Lynn Visioli, Lower Gwynedd, PA (US)

(73) Assignee: E. I. DU PONT DE NEMOURS AND COMPANY, Wilmington, DE (US)

(21) Appl. No.: 12/905,844

(22) Filed: Oct. 15, 2010

Related U.S. Application Data
(60) Provisional application No. 61/252,463, filed on Oct. 16, 2009.

(51) Int. Cl. A61L 15/22; A61B 19/08; B32B 3/26; B32B 5/24

(52) U.S. Cl. 602/43; 604/367; 128/849; 428/316.6

(57) ABSTRACT

Articles having zoned breathability such as personal hygiene articles are provided having controlled regional breathability with high MVTR regions and low MVTR regions. The zoned breathability articles comprise organic acid-modified ionomer resins.

This application claims priority to U.S. provisional application Ser. No. 61/252,463, filed Oct. 16, 2009; the entire disclosure of which is incorporated herein by reference.

The present invention relates to articles having zoned breathability such as personal hygiene articles.

### BACKGROUND OF THE INVENTION

Various types of personal hygiene articles are presently available for absorbing human discharge. Examples of these articles include baby diapers, feminine care products, incontinence garments and the like. Generally speaking, the basic structure of this class of garments requires a body-side liner pervious to aqueous liquid, an absorbent pad containing one or more layers for receiving and absorbing the discharge, and a backing member impervious to aqueous liquid for containing the discharge.

While some of these absorbent garments perform satisfactorily for their intended purpose, there remains the need to provide a more discreet absorbent garment that possesses improved comfort characteristics.

Some absorbent garments for absorbing and containing human discharge have typically been uncomfortable. For example, such absorbent garments may comprise flat sheets folded up into a diaper-like configuration having a film material that serves as liquid impervious outer cover. However, such film material lacks breathability, causing the absorbent garments to be hot and uncomfortable. The skin becomes overly hydrated by the aqueous liquids (for example, perspiration) trapped against the skin by the non-breathable film, resulting in skin occlusion.

Previous attempts at providing breathability for outer covers of personal hygiene articles include microporous membranes, which can provide controlled permeability to water vapor. Microporous membranes are films comprising liquid impermeable compositions in which microscopic defects or “pores” have been introduced, often by including filler that provides a gap in the film when the film is stretched. These microporous membranes may allow passage of water vapor through, while limiting passage of larger amounts of liquid water. Because of their structure, microporous membranes have some drawbacks. Along with the water vapor, other small odorous molecules may pass through the breathable barrier and create an unpleasant odor. Further, the pores may be sufficiently large enough to allow passage of bacteria and viruses through the microporous membrane.

Thus, it becomes apparent that a need exists for an absorbent garment that improves the containment characteristics of the absorbent garment while still being comfortable to wear as well as promoting skin wellness and skin dryness.

Monolithic films are “breathable” barriers in the sense that the film acts as a barrier to aqueous liquids and particulate matter but allows water vapor and air to pass through. By achieving and maintaining high breathability, it is possible to provide an article that is more comfortable to wear since the migration of water vapor through the fabric helps reduce and/or limit discomfort resulting from excess moisture trapped against the skin. Thus, such an article can potentially contribute to an overall improved skin wellness. Monolithic films also act as a barrier to bacteria and viruses and can provide an article or garment that reduces the contamination of the surroundings and the spread of infections and illness caused by the bacteria and viruses.

Accordingly, breathable films have become an important article of commerce, finding a wide variety of applications. For example, breathable films have been used as outer covers for personal hygiene articles such as diapers, training pants, incontinence garments, feminine hygiene products and the like. In addition, breathable films have likewise found use in protective apparel and infection control products such as surgical gowns, surgical drapes, protective workwear, wound dressings and bandages. Often breathable films are utilized as a multilayer laminate. The films can provide the desired barrier properties to the article while other materials laminated thereto can provide additional characteristics such as strength, abrasion resistance and/or softness and drapability. For example, fibrous webs such as nonwoven fabrics allow the laminate to retain its breathability and can provide additional strength as well as an article having a cloth-like feel. Thus, breathable film laminates can be used in a variety of applications including, for example, those described above.

Although the breathability provided by breathable films and/or laminates thereof is advantageous in many articles, there exist some situations where high breathability can be undesirable. For example, in absorbent personal care articles such as diapers or incontinence garments designed to absorb and contain aqueous liquid human exudates the breathable barrier and absorbent core generally work together to retain bodily fluids discharged into the garment. However, when fluid (aqueous liquid) is retained within the absorbent core significantly higher amounts of water vapor begin to pass through the breathable barrier. The increased amounts of water vapor passing through the outer cover can form condensate on the outer portion of the garment. The condensate is simply water but can be perceived by the wearer as leakage. In addition, the condensate can create a damp uncomfortable feel to the outer portion of the garment which is unpleasant for those handling the article.

The skin wellness and/or improved comfort benefits of breathable outer covers may not be achieved at areas directly adjacent to the portion of the absorbent core retaining considerable amounts of aqueous liquid (e.g. typically those areas of the central or crotch region of the personal hygiene article). Providing a breathable barrier which has less or limited breathability in such regions, while providing good breathability in the remaining regions, provides a garment with excellent wearer comfort yet which limits the potential for outer cover dampness or odor. Thus, a breathable barrier that provides either zoned or controlled regional breathability is highly desirable.

US Statutory Invention Registrations H1978 and H2011 describe monolithic films having controlled regional breathability with high moisture vapor transmission rate (MVTR) and low MVTR regions, made by selectively applying adhesive to the monolithic films. The films are used in absorbent undergarments, diaper training pants or the like, to provide desired absorbency and containment characteristics of absorbent garments and comfort during use.

U.S. Pat. No. 7,045,566 discloses moisture and gas permeable ionomeric films from blends of ionomers with an organic acid salt. U.S. Pat. No. 7,514,380 discloses articles comprising a selectively permeable membrane comprising an organic acid-modified ionomer.

However, there is a continuing need for preparation of films with areas of high breathability and areas of low breathability that are capable of lamination to additional materials to prepare articles such as absorbent undergarments to provide improved comfort during use. Polymeric compositions with improved breathability while retaining desired barrier properties would be desirable for preparing such films.

### SUMMARY OF THE INVENTION

This invention provides an article comprising at least one region of high vapor permeability and at least one region of low vapor permeability, wherein the region of high vapor permeability comprises a layer comprising, consisting essentially of, consisting of, or prepared from, an organic acid-modified ionomer composition comprising, consisting essentially of, or consisting of an ionomer and an organic acid or salt thereof wherein the ionomer has at least 60% of the acid moieties in the ionomer and organic acid are neutralized with an alkali metal and the organic acid-modified ionomer layer has a moisture vapor transmission rate (MVTR), measured according to ASTM F2298, of at least 800 g/m²/24 hours; and wherein the region of low vapor permeability has a MVTR that is at least 15% less than the MVTR of the first region.

This invention provides the article described above comprising

(a) a support layer;

(b) at least one region of high vapor permeability comprising a layer of the organic acid-modified ionomer composition overlying a first portion of the support layer; and

(c) at least one region of low vapor permeability having a MVTR that is at least 15% less than the MVTR of the first region overlying a second portion of the support layer wherein the first region and the second region do not overlay identical portions of the support layer.

The first region of the articles described above may comprise a monolithic film comprising the organic acid-modified ionomer composition. The articles disclosed above may be personal hygiene articles. Alternatively, the articles may be a multilayer structure suitable for use in preparing personal hygiene articles.

The first region may have MVTR in excess of about 2500 g/m²/24 hours and the second region has MVTR less than about 1500 g/m²/24 hours. Additionally and/or alternatively, the second region can have MVTR at least about 50% less than the MVTR of the first region. Further, the personal hygiene article can comprise a third region having a MVTR intermediate to those of the first and second regions.

### DETAILED DESCRIPTION OF THE INVENTION

All percentages, parts, ratios, etc., are by weight. When an amount, concentration, or other value or parameter is given as either a range, preferred range or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

“(Meth)acrylic acid” includes methacrylic acid and/or acrylic acid and “(meth)acrylate” includes methacrylate and/or acrylate.

The term “barrier” means a film, laminate or other fabric which is relatively impervious to the transmission of aqueous liquids and which has a hydrohead of at least about 50 mbar and in some applications greater than about 80 mbar, 150 mbar or even 200 mbar. Hydrohead as used herein refers to a measure of the aqueous liquid barrier properties of a fabric measured in millibars (mbar) as described below.

The term “breathability” refers to the MVTR of an area of fabric which is measured in grams of water per square meter per 24 hours (g/m.sup.2 /24 hours). The MVTR of a fabric is the water vapor transmission rate which, in one aspect, gives an indication of how comfortable a fabric would be to wear. MVTR can be measured as indicated below and the results are reported in grams/square meter/24 hours.

Monolithic film is a non-porous film. Rather than holes produced by a physical processing of the monolithic film, the film has passages with cross-sectional sizes on a molecular scale formed by a polymerization process. The passages serve as conduits by which water (or other liquid) molecules can disseminate through the film. Vapor transmission occurs through a monolithic film as a result of a concentration gradient across the monolithic film. This process is referred to as activated diffusion. As water (or other liquid) evaporates on the body side of the film, the concentration of water vapor increases. The water vapor condenses and solubilizes on the surface of the body side of the film. As a liquid, the water molecules dissolve into the film. The water molecules then diffuse through the monolithic film and re-evaporate into the air on the side having a lower water vapor concentration.

As this is mainly a diffusion-rate limited phenomenon, MVTR is a function of the type of polymer used in the monolithic film and the thickness of the monolithic film. As such, the permeability is selective in monolithic films. Permeability can be increased or decreased by changing the chemical or structural characteristics of the polymers used in the construction of the film.

A monolithic film provides an absolute barrier to liquids, bacteria, and viruses as no pores are present in the film. However, distortion of the passages within a monolithic structure can cause elongation or deformation which may enable viral pathogens to pass through the elongated opening of such passages. The liquid barrier properties of monolithic films are the result of the density of each type of monolithic film which prevents the passage of condensed liquids regardless of the viscosity or surface tension of the liquids. The liquid barrier properties are defined by burst strength, tensile properties, and abrasion resistance of the monolithic film as no liquid flow is possible unless the film ruptures.

Monolithic films can have the property of water resistance, surfactant insensitive, selective permeability, high water entry pressure, variable water swelling, good tear strength, and excellent odor barrier.

Monolithic films are inherently breathable. Because of that, the monolithic films do not require the addition of fillers and stretching to generate micro-porosity. The benefit of this is threefold. First, intact monolithic films are absolute barriers to all liquids (including alcohol), odor-causing molecules, bacteria, and viruses. The likelihood of defects within the monolithic film is reduced as holes are never intentionally introduced into the film.

And third, the elasticity of the film is not skewed by stretching and the excellent elastic properties of the polymer are fully maintained. Functional barrier and elastic films can be surprisingly thin, further enhancing breathability, thus, a low basis weight film can have excellent elastic properties and high breathability. Monolithic films are able to withstand high strain rates of being rapidly elongated to at least about 400% elongation. Micro-porous films shred under high strain rates.

A breathable monolithic film can be treated to create a breathable film having regions of varied breathability, which can be used as a backsheet for personal hygiene products. The term “backsheet” refers to the aqueous liquid impervious protective layer on the garment side of a personal hygiene product which prevents bodily exudates from escaping from the product. The variable breathability is achieved by coating various regions of the breathable film with materials having lower MVTR than the breathable film. The coating material may be any material that adheres to the breathable monolithic film when applied by a coating apparatus, thereby reducing the MVTR of the monolithic film where the coating has been applied.

The monolithic film comprises, consists essentially of, or is produced from a composition comprising or consisting essentially of an organic acid-modified ionomer.

The organic acid-modified ionomer can comprise, consist essentially of, or consist of one or more E/X/Y copolymers where E represents copolymerized units of ethylene, X represents copolymerized units of at least one C₃-C₈ α,β-ethylenically unsaturated carboxylic acid, and Y represents copolymerized units of a softening comonomer, or ionomers of the E/X/Y copolymers, wherein X is from about 3 to 35, 4 to 25, or 5 to 20, weight % of the E/X/Y copolymer, and Y is from 0 to about 35, 0.1 to 35, or about 5 to 30, weight % of the E/X/Y copolymer.

X includes unsaturated acids such as acrylic acid, methacrylic acid, maleic acid, fumaric acid, and itaconic acid; maleic (maleic half-esters) or fumaric monoesters including esters of C₁ to C₄ alcohols, such as, for example, methyl, ethyl, n-propyl, isopropyl, and n-butyl alcohols.

Softening comonomer can disrupt the crystallinity of an acid copolymer making the polymer less crystalline including alkyl(meth)acrylate where the alkyl groups have from 1 to 8 or 1 to 4 carbon atoms.

The composition can comprise, consist essentially of, or consist of the E/X/Y copolymer and one or more organic acids or salts thereof The organic acid or salt thereof can be present in the composition from 1 to 50 weight % and be selected from saturated or unsaturated monobasic or polybasic carboxylic acids having fewer than 36 carbon atoms, optionally substituted with from one to three substituents independently selected from the group consisting of C₁-C₈ alkyl, OH and OR¹, each R¹ is independently C₁-C₈ alkyl, C₁-C₆ alkoxyalkyl or COR²; and each R² is independently H or C₁-C₈ alkyl.

At least 60%, 70%, 80%, 90%, or even 100% of the acidic groups in the E/X/Y copolymer and the organic acid are nominally neutralized with metal ions to the corresponding salts, and the metal ions present in the mixture comprise a preponderance of alkali metal ions, preferably sodium or potassium ions, more preferably potassium ions.

Ethylene acid copolymers with high levels of acid (X) can be prepared by use of “co-solvent technology” as described in U.S. Pat. No. 5,028,674 or by employing somewhat higher pressures than those at which copolymers with lower acid levels can be prepared.

Specific acid copolymers include ethylene/(meth)acrylic acid copolymers. They also include ethylene/(meth)acrylic acid/n-butyl(meth)acrylate, ethylene/(meth)acrylic acid/iso-butyl(meth)acrylate, ethylene/(meth)acrylic acid/methyl(meth)acrylate, and ethylene/(meth)acrylic acid/ethyl(meth)acrylate terpolymers.

Other acid copolymers include ethylene/maleic acid and ethylene/maleic acid monoester dipolymers; and ethylene/maleic acid monoester/n-butyl(meth)acrylate, ethylene/maleic acid monoester/methyl(meth)acrylate, ethylene/maleic acid mono-ester/ethyl(meth)acrylate terpolymers.

Unmodified, melt processible ionomers can be prepared from acid copolymers such as ethylene/(meth)acrylic acid copolymers, by treatment with a basic compound capable of neutralizing the acid moieties of the copolymer.

Basic inorganic metal compound capable of neutralizing acidic groups in components may be provided by adding the stoichiometric amount of the basic compound calculated to neutralize a target amount of acid moieties in the acid copolymer and organic acid(s) in the blend (herein referred to as “% nominal neutralization” or “nominally neutralized”). Thus, sufficient basic compound is made available in the blend so that, in aggregate, the indicated level of nominal neutralization could be achieved. Nominal neutralization levels greater than 70, 80, or 90% of all acid moieties in the composition are preferred.

Basic compounds include compounds of alkali metals, such as lithium, sodium, potassium, or combinations of such cations. Preferred are sodium and potassium salts or combinations of sodium and potassium. Basic compounds of note include formates, acetates, nitrates, carbonates, hydrogencarbonates, oxides, hydroxides or alkoxides of the ions of alkali metals such as sodium hydroxide, potassium hydroxide, sodium carbonate and potassium carbonate.

The organic acids include saturated or unsaturated monobasic carboxylic acids optionally substituted with one to three substituents independently selected from the group consisting of C₁-C₈ alkyl, OH and OR¹, or polybasic carboxylic acids optionally substituted with from one to three substituents independently selected from the group consisting of C₁-C₈ alkyl, OH and OR¹.

Particularly useful organic acids include C₄ to less than C₃₆ (e.g., C₃₄), more particularly C₆ to C₂₆, and even more particularly C₆-C₂₂ acids. Monobasic carboxylic acids include acids having only one carboxylic acid moiety. Specific organic acids include, but are not limited to, caproic acid, caprylic acid, capric acid, lauric acid, palmitic acid, stearic acid, isostearic acid, behenic acid, erucic acid, oleic acid, and linoleic acid.

Examples of monobasic acids substituted with alkyl include isostearic acid and citronellic acid. Examples of monobasic acids substituted with hydroxy include glycolic acid, lactic acid, 3-hydroxybutyric acid, 2-hydroxyisobutyric acid, 2-hydroxycaproic acid, 6-hydroxycaproic acid, 10-hydroxydecanoic acid, 12-hydroxydodecanoic acid, 12-hydroxystearic acid, or combinations of two or more thereof.

Hydroxy-substituted organic acid includes derivatives wherein the H of the hydroxyl moiety is replaced by R¹.

The composition can also optionally comprise one or more non-ionomeric polymers. The non-ionomeric polymer can be present in the composition from about 0.1 to about 40 weight % of one or more non-ionomeric ethylene-containing or polypropylene-containing polymers.

Non-ionomeric polymers including ethylene-containing polymer, ethylene/vinyl acetate copolymer, ethylene/alky(meth)acrylate copolymer, propylene-containing polymer, or combinations of two or more thereof can provide better processibility, improved strength, and toughness. The composition may contain up to 35 (e.g., 0.1 to 35, 0.1 to 15, or 0.1 to 10) weight % of non-ionomeric polymer.

Ethylene-containing polymers include polyethylene homopolymers and copolymers such as high density polyethylene, low density polyethylene, linear low density PE, very low PE or ultra-low density PE, metallocene PE; ethylene propylene copolymers; ethylene/propylene/diene monomer terpolymers; and ethylene copolymers derived from copolymerization of ethylene and at least one comonomer selected from the group consisting of alkyl(meth)acrylate, vinyl acetate, carbon monoxide (CO), maleic acid anhydride and maleic anhydride derivatives, such as maleic diesters.

Polyethylene (PE) homopolymers and copolymers useful for the compositions described herein can be prepared by a variety of well known methods such as the Ziegler-Natta catalyst polymerization (U.S. Pat. No. 4,076,698 and U.S. Pat. No. 3,645,992), metallocene catalyzed polymerization, Versipol® catalyzed polymerization and by free radical polymerization.

The densities of suitable PE range from about 0.865 g/cc to about 0.970 g/cc.

Ethylene copolymers having small amounts of a diolefin component such as butadiene, norbornadiene, hexadiene and isoprene are also generally suitable. Terpolymers such as ethylene/propylene/diene monomer are also suitable.

Suitable polymers for ethylene-containing polymers may also include ethylene copolymers obtained from copolymerization of ethylene with at least one polar monomer. Such suitable copolymers include: ethylene/vinyl acetate copolymers, ethylene/acrylic ester copolymers, ethylene/methacrylic ester copolymers, ethylene/vinyl acetate/CO copolymers, ethylene/acrylic ester/CO copolymers, and/or mixtures of any of these.

The composition may comprise at least one ethylene/vinyl acetate copolymer including copolymers derived from the copolymerization of ethylene and vinyl acetate or copolymers derived from the copolymerization of ethylene, vinyl acetate and an additional comonomer. The relative amount of the vinyl acetate comonomer incorporated into ethylene/vinyl acetate copolymers can vary from a few weight percent up to as high as 45 weight percent of the total copolymer or even higher.

“Ethylene/alkyl(meth)acrylate copolymer” includes copolymers of ethylene and alkyl acrylates or alkyl methacrylates wherein the alkyl moiety contains from one to eight carbon atoms. Examples of alkyl acrylates include methyl acrylate, ethyl acrylate and butyl acrylate. “Ethylene/methyl acrylate” means a copolymer of ethylene and methyl acrylate. “Ethylene/ethyl acrylate” means a copolymer of ethylene and ethyl acrylate. “Ethylene/butyl acrylate” means a copolymer of ethylene and butylacrylate.

Preferably, the alkyl group in the alkyl(meth)acrylate comonomer has from one to eight carbon atoms and the alkyl(meth)acrylate comonomer has a concentration range of from 5 to 45 weight percent of the ethylene/alkyl(meth)acrylate copolymer, preferably from 10 to 35 weight %, more preferably from 10 to 28 weight %. Most preferably, the alkyl group in the alkyl(meth)acrylate comonomer is methyl, ethyl or n-butyl.

Ethylene/alkyl(meth)acrylate copolymers can be prepared by processes well known in the polymer art using either autoclave or tubular reactors. Because the processes are well known to one skilled in the art, their description is omitted herein for the interest of brevity.

Polypropylene polymers include homopolymers, random copolymers, block copolymers and terpolymers of propylene. Because polypropylene is well known to one skilled in the art, further description is omitted herein for the interest of brevity.

A melt-processible, modified ionomer blend can be produced by heating a mixture of the E/X/Y copolymer or ionomer, the organic acid or salt thereof, the basic compound and optionally the non-ionomeric copolymer. For example, the components of the composition can be mixed by melt-blending the individual components; and concurrently or subsequently adding a sufficient amount of a basic compound capable of neutralization of the acid moieties (including those in the acid copolymer and in the organic acid), preferably to nominal neutralization levels greater than 70, 80, 90%, to near 100%, or to 100% or above; and optionally adding an ethylene-containing or polypropylene-containing polymer.

Treatment of acid copolymers and organic acids with basic compounds in this manner (concurrently or subsequently), without the use of an inert diluent, to prepare the composition can avoid loss of processibility or properties such as toughness and elongation to a level higher than that which would result in loss of melt processibility and properties for the ionomer alone. For example, an acid copolymer blended with organic acid(s) can be nominally neutralized to over 80%, 90%, or to about 100% or to 100% without losing melt processibility. In addition, nominal neutralization to about 100% or to 100% reduces the volatility of the organic acids.

The acid copolymer(s) or unmodified, melt-processible ionomer(s) can be melt-blended with the organic acid(s) or salt(s) and other polymers in any manner known in the art. For example, a salt and pepper blend of the components can be made and the components can then be melt-blended in an extruder.

The melt-processible, acid copolymer/organic-acid-or-salt blend can be treated with the basic compound by methods known in the art. For example, a Werner & Pfleiderer twin-screw extruder can be used to treat the acid copolymer and the organic acid with the basic compound at the same time.

The compositions can comprise additional additives including plasticizers, stabilizers including viscosity stabilizers and hydrolytic stabilizers, antioxidants, ultraviolet ray absorbers, anti-static agents, dyes, pigments or other coloring agents, lubricants, processing aids, antiblock agents, release agents, and/or mixtures thereof. These additives may be present in the compositions from 0.01 to 15, 0.01 to 10, or 0.01 to 5 weight %. The optional incorporation of such ingredients into the compositions can be carried out by any known process. This incorporation can be carried out, for example, by dry blending, by extruding a mixture of the various constituents, by a masterbatch technique, or the like.

The organic acid modified ionomer composition has water vapor permeation value (WVPV) at least 4000, or at least 5000, g-mil/m²/24 h or higher. The highly moisture permeable compositions described herein allow preparation of articles having zoned breathability, such as personal hygiene articles, infection control articles and protective covers as described herein. The highly permeable compositions compared to previous permeable compositions (for example, those described in U.S. Statutory Invention Registration H1978) provide for layers with higher MVTR for improved performance for use in personal hygiene articles and infection control products. The compositions also provide more design flexibility for preparing films having the desired MVTR with desirable thickness and suitable mechanical properties.

The organic acid modified ionomer composition may be adhered to a support layer as described below to provide an article comprising at least one region of high vapor permeability and at least one region of low vapor permeability comprising

(a) a support layer;

(b) at least one region of high vapor permeability comprising a layer of the organic acid-modified ionomer composition overlying a first portion of the support layer; and

(c) at least one region of low vapor permeability having a MVTR that is at least 15% less than the MVTR of the first region overlying a second portion of the support layer wherein the first region and the second region do not overlay identical portions of the support layer.

For example, an organic acid-modified ionomer composition can be adhered, extrusion coated, or extrusion laminated to a portion of a support layer to provide a first region having high vapor permeability. A composition having at least 15% lower vapor permeability can be adhered to a second portion of the support layer to provide a second region having lower vapor permeability.

In many cases, it is convenient to supply the support layer as a continuous web and apply the organic acid-modified ionomer composition to the support layer web in a continuous process.

For coating the organic acid-modified ionomer composition onto a support layer, conventional extrusion coating processes may be used, for example as described in Extrusion Coating Manual, 4^{th} Edition, ed. by Thomas Bezigian, TAPPI Press, Atlanta, Ga., 1999. Similar processes can be used to coat other thermoplastic resins, with lower breathability that the organic acid-modified ionomer composition, to provide second regions having at least 15% lower MVTR than regions coated with the organic acid-modified ionomer composition.

In one aspect, the thickness (amount or percentage of coverage) of the organic acid-modified ionomer composition coating layer can be varied in order to modify the breathability of the corresponding region of the support layer. Varying the thickness of the coating layer can provide varied levels of breathability extending in the machine direction, wherein thicker layers have reduced breathability compared to a reference thickness. For example, coating a thicker layer of the composition in the center portion of the support layer compared to the region(s) adjacent to at least one opposed edge of the support layer web provides a central region having reduced breathability compared to the region(s) adjacent to the opposed edges.

In another embodiment, the organic acid-modified ionomer composition can be applied to a portion of the support layer web in a first region and a second composition other than the organic acid modified composition having at least 15% lower vapor permeability can be applied to a different portion of the support web to provide a second region having lower permeability.

For example, the organic acid-modified ionomer composition can be applied in a continuous pattern in the machine direction of the support layer web in a region adjacent to least one opposed edge of the support layer web. A second composition other than the organic acid modified composition having at least 15% lower vapor permeability can be applied to the center portion of the support layer web, creating a zoned breathability article having a highly breathable region adjacent to at least one opposed edge of the support layer and a central second region of reduced breathability.

Alternatively, the application of the organic acid-modified ionomer composition can be non-contiguous in the sense that it is applied in a broken pattern. For example, the coating operation can be turned on and off as the support web passes through the coating machine, so that coated regions are spaced out in the machine direction. The center portion of the support layer web may be treated with either an additional coating of the organic acid-modified ionomer composition or another composition having lower breathability. The treatment of a support web as such creates a first region and second region in which the first region has greater breathability than second region. The treated areas may be generally rectangular or may have more complex shapes. A mask or stencil may facilitate coating of the organic acid-modified ionomer composition onto the support web in a desired shape.

The treated regions of the support web may extend at least 3 cm in the machine direction (the direction of travel of the continuous web, MD) and transverse direction (TD) and more desirably at least 5 cm×5 cm in the MD and TD. Further, the treated regions of the surface can extend at least 10 cm in either the MD or TD, depending on the use for which the article of zoned breathability is intended.

The treated regions may desirably comprise from about 5% to about 90% of the overall area of the support layer. Optionally, the treated regions can comprise a plurality of regions of intermediate and low breathability. The regions of low and intermediate breathability desirably form a single contiguous area and which can, in one aspect, be disposed about the central portion of the support web. However, the treated regions can comprise several non-contiguous regions and need not be centered on the breathable film.

A third region of breathability having breathability intermediate between the first region of high breathability and a second region of lower breathability can be prepared using similar techniques. The first region can comprise about 1 to about 99% of the film and the second region can comprise about 1 to about 99% of the film. Any additional region can be in the same percentage range and the areas of the first and second regions can be accordingly reduced.

Desirably, the first region, the second region and third region when present are contiguously applied to the support web.

Alternatively, the organic acid-modified ionomer composition can be formed or incorporated into films by known techniques such as casting the polymer composition onto a flat surface or into a film, extruding the molten polymer composition through an extruder to form a cast film, or extruding and blowing the polymer composition film to form an extruded blown film. These highly permeable monolithic films can be used to prepare articles of zoned breathability.

Suitable monolithic films include breathable monolithic films having a MVTR of at least 800 g/m²/24 hours, and more desirably having a MVTR in excess of 1500 g/m²/24 hours, 1800 g/m²/24 hours, 2500 g/m²/24 hours, 3500 g/m²/24 hours or higher. Desirably, the breathable monolithic film substrate has a MVTR between about 2000 g/m²/24 hours and about 7000 g/m²/24 hours.

The films can have a thickness of from 1 to 2500 μm, with the preferred thickness for many film applications being about 10 to 250 μm thick, alternatively less than 150 μm thick, or less than 50 μm thick, preferably 35 to 125 μm thick, or 10 to 35 μm thick. The MVTR of these films can be about 10 Kg/m²/24 hours or higher for a 50-micron thick continuous film.

Preferably, the film has a hydrohead of at least about 50 mbar.

Suitable films can also include multilayer films having at least one monolithic layer comprising an organic acid-modified ionomer composition as described above.

The organic acid modified compositions and films thereof described herein provide an improved breathable absorbent garment having improved comfort characteristics. Once the breathable monolithic film has been formed, the monolithic film can be incorporated into personal hygiene articles to provide a first region of high breathability. A second region having low vapor permeability such as a MVTR that is at least 15% less than the MVTR of the first region is also provided.

For example, the breathable monolithic film may be joined with one or more additional layers. Desirably the monolithic film is attached to a pliable support layer capable of being laminated to the monolithic film such as, for example, a pliable fibrous, film and/or foam material. The monolithic film may also be attached to an absorbent layer for receiving and absorbing fluids.

The support layer, described in greater detail below, can be attached or laminated to the monolithic film by adhesive bonding. The monolithic film and support layer may be bonded with an adhesive sprayed via a standard meltblown die to the support layer and/or monolithic film.

It is desirable that the material used as an adhesive provides good adhesion to both the breathable film and an additional material (including an additional layer of the breathable film) in order to bond the breathable film to the additional material. Such coating materials may be used to prepare multilayer structures. As described in more detail below, the manner of application of such a single coating material may have a bearing on its adhesive function while limiting its impact on the breathability of the monolithic film described herein. An example adhesive coating that may be used is a styrene block copolymer material EASYMELT® 34-5610 from National Starch and Chemical Company in Bridgewater, N.J.

To maintain the high breathability of the monolithic film, the adhesive material is preferably present as a discontinuous layer, such as a series of adhesive dots that cover for example about 10 to about 40 percent of the area of the film. When applied in a discontinuous or open pattern, the adhesive has minimal effect on the breathability of the monolithic film, but can be used as an adhesive layer used to attach the various components of product into which the monolithic film is incorporated for construction of multilayer structures and protective articles. The adhesive may be applied to the monolithic film in an open patterned application (for example, using a Nordson Control Coat CC-200 available from the Nordson Corporation at Norcross, Ga.). The adhesive can be pattern-applied over the entire area of the monolithic film or it can be pattern-applied only in the areas where adhesion of the breathable monolithic film to the support layer is desired (for example near the edges of the shaped monolithic film). The adhesive layer may be applied in amounts from about 1 g/m² to about 7 g/m², or from about 2 g/m² to about 5 g/m², such as 3.2 g/m².

Depending on how the personal hygiene article is desired to be constructed, the adhesive may be applied to the body-side surface of the monolithic film as it is incorporated into absorbent garments, or the adhesive coat layer may be applied to the garment-side surface of the monolithic film, or it may be applied to both body-side and garment-side surfaces of the monolithic film to facilitate construction of various articles. When the monolithic film is incorporated into a breathable absorbent garment, the body-side surface of the monolithic film refers to the surface of the monolithic film that may face toward the wearer and the garment side surface of the monolithic film refers to the surface of the monolithic film that may face away from the wearer, toward the wearer's clothes.

In addition to use of an adhesive, the support layer and monolithic film may be alternatively adhered using thermal bonding, RF welding, ultrasonic bonding or other means known in the art laminated via thermal point bonding. The monolithic film may be prepared by extrusion coating the breathable organic acid modified ionomer composition to a substrate or support layer.

Ultrasonic bonding means a process performed, for example, by passing the fabric between a sonic horn and anvil roll as illustrated in U.S. Pat. No. 4,374,888.

Point bonding means bonding one or more layers of fabric at numerous small, discrete bond points. For example, thermal point bonding generally involves passing one or more layers to be bonded between heated rolls such as, for example an engraved pattern roll and a smooth calender roll. The engraved roll is patterned in some way so that the entire fabric is not bonded over its entire surface, and the anvil roll is usually flat. As a result, various patterns for engraved rolls have been developed for functional as well as aesthetic reasons. One example of a pattern has points and is the Hansen Pennings or “H&P” pattern with about a 30% bond area when new and with about 200 bonds/square inch as taught in U.S. Pat. No. 3,855,046.

A second composition having at least 15% lower breathability can be adhered to the support layer to provide an article having zoned breathability.

For example, a film of the organic acid modified ionomer composition can be adhered to a support layer in a region adjacent to at least one edge of the support layer and a second film having at least 15% lower breathability can be adhered in a second region in the center portion of the support layer web.

In other embodiments, the monolithic film can be incorporated into an article having zoned breathability, such as a personal hygiene article, as a “patch” in which a shaped portion of the monolithic film is adhered to the other components of the article in a region wherein a first region of high breathability is desired. The shaped portion of monolithic film may be adhered to the support layer web in registered, spaced out fashion so that individual articles having zoned breathability can be cut from the continuous web of support layer. For example, a film made from a composition with low breathability may be adhered to the center portion of a support layer web to provide a region of low breathability, and shaped portions of the highly breathable organic acid modified ionomer monolithic film may be adhered to the support web. The shaped portions may be adhered to the support web in a portion adjacent to at least one opposed edge of the support web. Alternatively, shaped portions of the monolithic film may be adhered across the center portion of the support web and to at least one portion of the support web adjacent to the edge of the support web.

A mixture of coating and film adhesion may also be employed to prepare articles with zoned breathability. For example, a support web may be extrusion coated with a composition having lower breathability in a central region of the support web and a film of the organic acid modified ionomer composition may be adhered to the support web near at least one opposed edge of the support web. The film may be applied to the support web as a continuous film or shaped portions of film may be applied.

Exemplary fibrous layers include, but are not limited to, nonwoven webs, multilayer nonwoven laminates, scrims, woven fabrics, slit films and/or other like materials. Desirably the support fabric comprises one or more layers of spunbonded and/or meltblown fiber webs including, but not limited to, monocomponent spunbond fiber webs, multicomponent spunbond fiber webs, split fiber webs, multilayer nonwoven laminates, bonded carded webs and the like. Typically, these fibrous layers are highly breathable and do not impair the breathability of the monolithic film when attached to the monolithic film. Generally, the composition of the fibrous layer may be selected to achieve the desired properties, i.e. hand, aesthetics, tensile strength, cost, abrasion resistance, hook engagement, etc. It is understood that the bonding means used to attach the fabric layer to the monolithic film cannot impair the breathability of the monolithic film. This concern is not as great in areas where reduced MVTR is desired.

“Nonwoven” fabric or web means a web having a structure of individual fibers or threads which are interlaid, but not in an identifiable manner as in a knitted or woven fabric. Nonwoven fabrics or webs have been formed by many processes such as for example, meltblowing processes, spunbonding processes, hydroentangling, air-laid and bonded carded web processes.

Spunbond fibers are small diameter fibers of molecularly oriented polymeric material. Spunbond fibers may be formed by extruding molten thermoplastic material as filaments from a plurality of fine, usually circular capillaries of a spinneret with the diameter of the extruded filaments then being rapidly reduced as described in, for example, in U.S. Pat. Nos. 4,340,563; 3,692,618; 3,802,817; 3,338,992 and 3,341,394; 3,502,763; 3,542,615; 5,382,400 and 5,759,926. Spunbond fibers are generally not tacky when they are deposited onto a collecting surface and are generally continuous.

Meltblown fibers are fibers of polymeric material that are generally formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter. Thereafter, the meltblown fibers can be carried by the high velocity gas stream and are deposited on a collecting surface to form a web of randomly dispersed meltblown fibers. Such a process is disclosed, for example, in U.S. Pat. No. 3,849,241. Meltblown fibers may be continuous or discontinuous, are generally smaller than 10 microns in average diameter, and are generally tacky when deposited onto a collecting surface.

A multilayer nonwoven laminate is a laminate of two or more nonwoven layers such as, for example, wherein some of the layers are spunbond and some meltblown such as a spunbond/meltblown/spunbond (SMS) laminate. Examples of multilayer nonwoven laminates are disclosed in U.S. Pat. Nos. 4,041,203; 5,178,931 and 5,188,885. Such a laminate may be made by sequentially depositing onto a moving forming belt first a spunbond fabric layer, then a meltblown fabric layer and last another spunbond layer and then bonding the laminate such as by thermal point bonding as described below. Alternatively, the fabric layers may be made individually, collected in rolls, and combined in a separate bonding step.

The term “monocomponent” fiber refers to a fiber formed from one or more extruders using only one polymer. This is not meant to exclude fibers formed from one polymer to which additives have been added. The term “multicomponent fibers” refers to fibers which have been formed from at least two polymers extruded from separate extruders but spun together to form one fiber. Multicomponent fibers are also sometimes referred to as conjugate or bicomponent fibers. The polymers of a multicomponent fiber are arranged in substantially constantly positioned distinct zones across the cross-section of the fiber and extend continuously along the length of the fiber. The configuration of such a fiber may be, for example, a core/sheath arrangement wherein one polymer is surrounded by another or may be a side by side arrangement, a pie arrangement or an “islands-in-the-sea” type arrangement. Multicomponent fibers are taught in U.S. Pat. Nos. 5,108,820; 4,795,668 and 5,336,552. Conjugate fibers and methods of making them are also taught in U.S. Pat. No. 5,382,400 and may be used to produce crimp in the fibers by using the differential crystallization properties of the two (or more) polymers. The fibers may also have various shapes such as those described in U.S. Pat. Nos. 5,277,976; 5,466,410 and 5,069,970 and 5,057,368.

Biconstituent or multiconstituent fibers are fibers which have been formed from at least two polymers extruded from the same extruder as a blend. The Biconstituent fibers do not have the various polymer components arranged in relatively constantly positioned distinct zones across the cross-sectional area of the fiber and the various polymers are usually not continuous along the entire length of the fiber, instead usually forming fibrils or protofibrils which start and end at random. Bicomponent and biconstituent fibers are discussed in U.S. Pat. No. 5,294,482 and in the textbook Polymer Blends and Composites by John A. Manson and Leslie H. Sperling, copyright 1976 by Plenum Press, a division of Plenum Publishing Corporation of New York, ISBN 0-306-30831-2, at pages 273 through 277.

“Scrim” means a lightweight fabric used as a backing material. Scrims are often used as the base fabric for coated or laminated products.

Further, the fibrous layer can also be treated, for example, by embossing, hydroentangling, mechanically softening, printing or treated in another manner in order to achieve additional desired characteristics. In one embodiment the outer layer may comprise about a 10 g/m² to about 68 g/m² web of spunbonded polyolefin fibers and even more desirably a 10 g/m² to about 34 g/m² web of such fibers.

The articles having controlled regional breathability can be used with a wide variety of products or as components of products such as, for example, in personal hygiene articles, infection control products, protective covers, garments and the like.

Personal hygiene articles include personal hygiene oriented items such as diapers, training pants, absorbent underpants, adult incontinence products, feminine hygiene products, and the like. In general, these are articles which are worn to contain bodily discharge(s) from the wearer.

Infection control products include medically oriented items such as surgical gowns and drapes, face masks, head coverings like bouffant caps, surgical caps and hoods, footwear like shoe coverings, boot covers and slippers, wipers, garments like lab coats, coveralls, aprons and jackets, patient bedding, stretcher and bassinet sheets and the like. In general, these are articles are used to protect the user from bodily discharge(s) from other individuals. Other infection control products include wound dressings, bandages, sterilization wraps, and the like that contain bodily discharges and/or protect the wound from external contamination.

A protective cover includes a cover for vehicles such as cars, trucks, boats, airplanes, motorcycles, bicycles, golf carts, etc., covers for equipment often left outdoors like grills, yard and garden equipment (mowers, roto-tillers, etc.) and lawn furniture, as well as floor coverings, table cloths, picnic area covers, tents and the like.

When incorporated into a personal hygiene article, the breathable organic acid modified ionomer composition (for example in the form of a monolithic film) provides a first region of high breathability. A second region of lower breathability may be provided by adhering a film having lower vapor permeability in another area of the personal hygiene article to provide a second region of low vapor permeability has a MVTR that is at least 15% less than the MVTR of the first region.

In some embodiments, the article of zoned breathability is combined with an absorbent layer to for receiving and absorbing fluids. In many cases, the regions more or less coextensive with the absorbent pad or layer are of lower breathability (i.e. the second regions as described above). The absorbent pad need not cover the entire second region and that the absorbent pad may overlap onto a portion of the first region. Often, the portion of the absorbent pad that has the highest aqueous liquid loading may be positioned over the second region.

The breathable absorbent garment may further provide an absorbent pad disposed between a breathable backing member comprising the organic acid modified ionomer composition described herein and a body-side liner. The breathable absorbent garment may also include an elasticized design that also facilitates the formation of the crotch section, as well as an effective seal between the garment and the wearer, whereby the garment is comfortable to wear.

As a particular example, a monolithic film comprising the organic acid modified ionomer composition can be readily converted and incorporated within a breathable barrier of a diaper or incontinence garment whereby regions of reduced breathability extend along the central portion or crotch of the diaper, optionally in combination with an absorbent pad, as discussed above. The regions more or less coextensive with the absorbent pad may be of lower breathability, while regions typically of higher breathability extend along the outer portions or “ears” of the garment where the absorbent pad is not present to maximize dryness or skin health. Alternate embodiments include garments with a shaped monolithic backing member having high breathability, a region of reduced breathability and an absorbent pad which have leg cutouts typically included for improved fit and comfort. The size and/or shape of the absorbent pad may coincide with the size and/or shape of the second region.

Additional embodiments of absorbent garments that can be prepared from the zone breathability films described herein include garments constructed according to methods described in U.S. Statutory Invention Registration H2011.

In another example, the zoned breathability monolithic films may be used in surgical gowns. It is believed that the regions of reduced breathability, particularly areas where breathability has been significantly or almost completely reduced, may provide improved barrier properties. For example, areas of reduced breathability are believed to provide improved barrier properties to blood-borne pathogens. Thus, surgical gowns can be fabricated employing low breathability regions within high risk areas, such as the forearms or front of the gown, and higher MVTR regions within lower risk areas. The monolithic film can also be utilized in numerous other applications employing breathable barrier fabrics.

Wound dressings, bandages, sterile wraps and the like may optionally have pressure-sensitive adhesive applied to the body-side surface of the organic acid modified ionomer film in regions that do not include reduced breathability and/or absorbent pad such as the margins or extended flaps to enable fastening to the user.

The following Examples are presented to more fully demonstrate and illustrate, but are not meant to unduly limit the scope of, the invention.

### EXAMPLES

### Test Methods

Hydrohead: A measure of the liquid barrier properties of a fabric or film is the hydrohead test, which determines the height of water or amount of water pressure (in millibars) that the fabric may support before aqueous liquid passes through. A film with a higher hydrohead reading indicates it has a greater barrier to aqueous liquid penetration than a fabric with a lower hydrohead. The hydrohead can be performed according to Federal Test Standard 191A, Method 5514.

MVTR: Moisture vapor transmission rate (MVTR) is the rate of transmission through the entire thickness of a film and is inversely proportional to the thickness of the film. The WVPV is normalized to a film of 1-mil thickness and is an indicator of the inherent permeability of the composition(s) used to prepare the film. For film samples, water vapor permeation tests are conducted on a Mocon Permatran-W® 101K, following ASTM D6701-01 or ASTM F2298, at 37.8° C. WVPV on film samples are reported in g-mil/m²-24h while MVTR are reported g/m²-24h. The composition or membrane has WVPV at least 4000, or at least 5000, g-mil/m²/24h.

High MVTR values can result in condensation of water vapor on the outer surface of an absorbent garment. This is perceived as leakage by many consumers. Evaluation of this can be conducted according to methods described in U.S. Statutory Invention Registration H1978.

High MVTR levels in nonabsorbent areas of a disposable garment increase wearer comfort. Evaluation of this can be conducted according to methods described in U.S. Statutory Invention Registration H1978.

The ability of moisture and heat to permeate through fabric is a significant factor in determining how comfortable a garment may be. Heat can be transferred through a fabric in two ways: dry heat transfer and/or moisture-assisted heat transfer. From the dry and wet heat transfer rate measurements, the permeability index (Im), can be calculated. A method for determining material “breathability,” or evaporative resistance, uses a Guarded Sweating Hotplate Test according to ASTM F1868, ISO 11092. This test measures the dry and wet heat transfer rates of a material. A particular variation of this test used to evaluate performance of materials for use in diapers is described in U.S. Statutory Invention Registration H1978. The test can also be used to evaluate how warm or cool a material feels to the touch and the thermal conductivity of materials.

Wet Heat Transfer represents the amount of heat that is transferred from the skin through the fabric to the outside environment with the assistance of moisture. The larger the wet heat transfer value, the more heat may be lost or transferred through the fabric with the assistance of moisture. This test is appropriate for the measurement of heat transfer in most situations where the wearer would perspire.

ASTM 1670 and ISO 16603 are test methods commonly used to show resistance to liquid water flow. In these tests, a specimen is subjected to a body fluid simulant for a specified time and pressure sequence. A visual observation is made to determine when, or if, penetration occurs.

Im or Permeability Index is the ratio of the thermal and evaporative resistance of the fabric to the ratio of thermal and evaporative resistance of air. As the value approaches 1, the less resistant or more air-like the fabric is. For example, a lightweight, loosely woven fabric would have a larger Im value than TYVEK®. (Differences as small as 0.01 can be perceived.)

High MVTR levels in certain areas of a disposable garment increase wearer skin wellness by reducing skin occlusion and excessive hydration of the skin. Evaluation of this can be conducted according to methods described in U.S. Statutory Invention Registration H1978.

In order to illustrate the moisture permeance associated with a film layer involving a selectively permeable composition as described herein, extrusion cast films were prepared from the materials listed below.

### Materials Used

Ionomer 1 was a terpolymer comprising ethylene, n-butyl acrylate (23.5 weight %) and methacrylic acid (9 weight percent), neutralized to 52% (nominally) with sodium using sodium hydroxide, having a MI of 1.
Ionomer 2 was a copolymer comprising ethylene and methacrylic acid (19 weight percent), neutralized to 37% (nominally) with sodium using sodium hydroxide, having a MI of 2.6.
EAC-1 was a terpolymer comprising ethylene, n-butyl acrylate (23.5 weight %) and methacrylic acid (9 weight percent), having a MI of 25. This is the base resin for Ionomer 1 prior to neutralization.
EAC-2 was a dipolymer comprising ethylene, and methacrylic acid (19 weight percent), having a MI of 300.
HSA: 12-hydroxystearic acid commercially supplied by ACME-Hardesty Co.
ISA: Iso-stearic acid commercially supplied by Arizona Chemical.
BEH: behenic acid commercially supplied by Uniqema.
ABA: A mixture containing 90 weight % of a mixture of arachidic acid and behenic acid with 6 weight % C₁₈ acids and 4 weight % other acids commercially available under the tradename Hystrene® 9022 from Chemtura.
Base MB-1: A blend of 59.5 weight % Na₂CO₃ in an ethylene/methylacrylic acid (10 weight %) copolymer with MI of 450 g/10 minutes.
Base MB-2: A blend of 50% K₂CO₃ in an E/methyl acrylate (24 weight %) copolymer with MI of 20 g/10 minutes.
Base MB-3: A 50% K₂CO₃ solution in water.
PE-1: low density polyethylene available under the designation DPE 1640 from DuPont Performance Elastomers

### Examples 1-4

Employing a Werner & Pfleiderer twin-screw extruder, ionomer 3 was melt blended with 40 weight % of potassium stearate and additional potassium hydroxide to neutralize the composition to nominally 100% neutralization to provide Example 2. Other examples in Table 1 were prepared similarly, using the indicated ionomer or ethylene acid copolymer blended with the indicated fatty acid modifier and neutralized to 100% nominal neutralization with the potassium hydroxide.

TABLE 1

| Example | Ionomer | Modifier (wt. %)* | WVPV (g-mil/m²-24 h) |
|---|---|---|---|
| 1 | Ionomer 2 | K stearate (40%) | 5,387 |
| 2 | Ionomer 1 | K stearate (40%) | 5,279 |
| 3 | Ionomer 2 | K iso-stearate (20%) | 10,290 |
| 4 | Ionomer 2 | K iso-stearate (40%) | 78,535 |

*Examples were brought to 100% nominal neutralization with KOH.

### Examples 5-9

Additional film examples were prepared by extrusion casting.

TABLE 2

| Example | Acid copolymer (wt %) | Modifier (wt %) | Neutralizing agent (wt %) | EMA-1 (wt %) | WVPV (g-mil/m²-24 h) |
|---|---|---|---|---|---|
| 5 | EAC-2 (70.63) | BEH (7.85) | MB-2 (21.25) | 0 | 9504 |
| 6 | EAC-2 (49.57) | BEH (21.24) | MB-2 (21.32) | 7.87 | 11401 |
| 7 | EAC-2 (77.57) | HSA (8.62) | K₂CO_{3 (}13.81) | 0 | 9844 |
| 8 | EAC-2 (67.81) | ISA (16.95) | K₂CO₃₍ (15.24) | 0 | 32145 |
| 9 | EAC-1 (72.75) | ISA (18.19) | K₂CO₃₍ (9.06) | 0 | 10485 |

### Examples 10-16

The indicated materials were melt-blended in a twin-screw extruder at 20 lb/h (about 9 kg/h) throughput rate to provide compositions summarized in Table 3 below. Unless noted otherwise, the compositions were cast into films of 2 to 2.5 mils (except that examples 14-16 were 4 mils) thickness via a 28 mm W&P twin screw extruder.

TABLE 3

| Example | Polymer (wt %) | EMA-1 (wt %) | Modifier (wt %) | Neutralizing Agent (wt %) | MVPV (g-mil/m²-24 h) |
|---|---|---|---|---|---|
| 10 | Ionomer 2 (72.57) | 0 | ISA (18.14) | KOH (9.29) | 53920 |
| 11 | Ionomer 2 (83.3) | 0 | HSA (9.3) | KOH (7.4) | 5188 |
| 12 | Ionomer 2 (69.13) | 12.84 | HSA (3.63) | MB-2 (14.39) | 4219 |
| 13 | EAC-2 (75.38) | 0 | HSA (3.14) | KOH | 10333 |
| 14 | EAC-2 (57.72) | 20.33 | HSA (3.25) | MB-2 (18.70) | 4415 |
| 15 | EAC-2 (78.40) | 0 | HSA (3.27) | MB-3 (11.15) & MB-1 (7.19) | 5079 |
| 16 | EAC-2 (59.71) | 21.03 | HSA (3.36) | MB-3 (9.67) & MB-1 (6.22) | 5006 |

Employing a Werner & Pfleiderer twin-screw extruder, a composition containing 80 weight % of EAC-2 and 20 weight % of ABA was nominally neutralized to 93-95% with potassium hydroxide (Example 17). Example 17 was extruded through a film die to prepare a cast film with 2-mil thickness and the properties summarized in Table 4.

TABLE 4
Properties of Example 17

| | MD | TD | Average |
|---|---|---|---|
| 2% Tensile Modulus | | | |
| (psi) | 25400 | 18300 | 21850 |
| Elmendorf Tear-notched (ASTM1922) | | | Unnotched |
| (g/mil) | 9.07 | 20.6 | 14.84 |
| (g/mm) | 348 | 790 | 569 |
| Tensile properties (2 inch/min) | | | |
| Tensile strength (psi) | 1600 | 1100 | 1350 |
| Elongation at break (%) | 290 | 149 | 219.5 |
| WVTR (mil-g/m²-day) | 12721 | | |

### Example 18

A monolithic film of Example 17 is cut into 12-cm widths and portions are laminated to a 50-cm wide web of non-woven fabric support layer in two 12-cm wide areas, each area starting 3 cm from an edge of the support layer and extending toward the center of the support layer web. A second film (2-mil thickness and 24 cm wide) of PE-1 is laminated to the center portion of the support web so that its edges partially overlap with the edges of the Example 17 monolithic films. The overlaps ensure that there are no gaps between the films that would allow liquid to pass through. The resulting laminate has first regions of high breathability in the areas adjacent to the edges of the support layer web and a second region of low breathability in the center portion of the web contiguous with the first regions. This laminate can be used to form an outer cover for a personal hygiene article.

1. An article comprising at least one first region and at least one second region wherein
the article is diaper, training pants, absorbent underpants, adult incontinence product, feminine hygiene product, an infection control product including surgical gowns and drapes, face masks, head coverings, footwear, wipers, patient bedding, stretcher and bassinet sheets, wound dressing, bandage, sterilization wrap, table cloth, picnic area, tent, tarpaulin, garment, floor covering, or a protective cover for an article selected from the group consisting of a vehicle, equipment often left outdoors, and lawn furniture;
the organic acid-modified ionomer comprises, or is produced from, one or more E/X/Y copolymers and an organic acid or salt thereof;
E represents copolymerized units of ethylene, X represents copolymerized units of at least one C₃-C₈ α,β-ethylenically unsaturated carboxylic acid, and Y represents copolymerized units of a softening comonomer, or ionomers of the E/X/Y copolymers, wherein X is from about 3 to 35 weight % of the E/X/Y copolymer, and Y is from 0 to about 35 weight % of the E/X/Y copolymer;
at least 60% of the acid moieties in the E/X/Y copolymer and organic acid are neutralized with an alkali metal;
the first region has a moisture vapor transmission rate (MVTR), measured according to ASTM F2298, of at least 800 g/m² /24 hours; and
the second has a MVTR that is at least 15% less than the MVTR of the first region.

2. The article of claim 1 wherein at least 60% of the acid moieties in the E/X/Y copolymer and organic acid are neutralized with an alkali metal.

3. The article of claim 2 wherein the first region has MVTR in excess of about 2500 g/m²/24 hours and the second region has MVTR less than about 1500 g/m²/24 hours.

4. The article of claim 2 wherein the second region has MVTR at least about 50% less than the MVTR of the first region.

5. The article of claim 2 further comprising a third region having MVTR intermediate to those of the first and second regions.

6. The article of claim 2 wherein the first region has a water vapor permeation value of at least 4000 g-mil/m²/24 h.

7. The article of claim 6 wherein the first region has a water vapor permeation value of at least 5000, g-mil/m²/24 h.

8. The article of claim 1 further comprising a support layer wherein
at least one of the first regions or a portion of the first region overlays a first portion of the support layer;
at least one of the second regions or a portion of the second region overlays a second portion of the support layer; and
the first region and the second region do not overlay identical portions of the support layer.

9. The article of claim 8 wherein the support layer comprises at least one layer comprising a material selected from the group consisting of a pliable fibrous film or a pliable fibrous foam.

10. The article of claim 8 further comprising an absorbent layer for receiving and absorbing fluids.

11. The article of claim 10 wherein the absorbent layer is attached to the support layer.
